(19) United States
(12) Patent Application Publication
(10) Pub. No.: US 2003/0017216 A1
(43) Pub. Date: Jan. 23, 2003

(54) Isolation of herbal and cannabinoid medicinal extracts

(76) Inventors: Robert Gustav Schmidt, Petaluma, CA (US); Charles Edward Coco JR., Pleasanton, CA (US)

Correspondence Address: William R. Hyde, 1785 Aladdin Road, Colville, WA, 99114, US

(21) Appl. No.: 09/911,900

(22) Filed: Jul. 23, 2001

(51) Int. Cl. A61K 35/78

(52) U.S. Cl. 424/725

(57) ABSTRACT

Extracts of Cannabis are prepared by processes utilizing a time-sensitive partial extraction wherein the solvent is allowed to contact the Cannabis plant material for a period of time less than that needed to reach an equilibrium of dissolved cannabinoids in solvent. Such time-sensitive selective partial extraction may also be utilized with other medicinal plants having elevated levels of soluble therapeutic components in plant surface structures or near-surface structures.

### BACKGROUND OF THE INVENTION

1. Field of the Invention

The present invention generally relates to the preparation of herbal extracts and oils. More particularly, the present invention relates to the extraction and purification of Cannabis oils.

2. Description of the Related Art

Cannabis and hemp have played important roles in most societies. The active ingredients in Cannabis species, including Cannabis indica and C. sativa, have been found to have medicinal properties including relief of symptoms of various diseases and conditions. The plant itself contains some 400 cannabinoids, each of which may have some therapeutic potential. Cannabinoids are compounds with 21 carbon atoms and carboxylic acids, analogs and transformation products of the 21-carbon compounds (the carboxylic acids are particularly prevalent in living cells and fresh plant product until aging, drying and/or heat decarboxylate them). Although the relative percentage of cannabinoids in Cannabis plants varies greatly with genetic and environmental factors, major constituents typically include the tetrahydrocannabinols (collectively referred to as THC), cannabidiol (CBD) and cannabinol (CBN) along with minor constituents such as cannabichromene (CBC). Medical grade Cannabis plants are typically high in THC, with varying levels of CBD.

Medicinal applications for Cannabis plants and extracts include analgesia for acute and chronic pain, antiemetic therapy for nausea and vomiting (especially chemotherapy and radiation induced nausea and vomiting), appetite stimulation including malnutrition and wasting syndrome in AIDS patients and malnutrition in cancer patients, anti-spasticity, particularly in multiple sclerosis, muscular dystrophy and spinal cord injury patients, movement disorders such as Parkinson's disease, Huntington's disease and Tourette's syndrome, epilepsy, glaucoma (including intraocular pressure and vision improvement), alleviation of depression, anxiety and stress, migraine headaches, arthritis and rheumatism, and easement of symptoms of various other chronic illnesses and conditions. While research has not yet identified all of the compounds responsible for these pharmacological effects, clinical experience has shown that the use of the whole plant material or extracts thereof presents numerous advantages as compared to isolated or synthesized compounds such as THC (Marinol).

Historic delivery methods such as smoking the vegetable matter have been shown to produce potentially toxic substances and adverse effects on the respiratory system as results from burning or smoking any substance. Therefore as a cannabinoid drug delivery system, marijuana cigarettes are not ideal in that they deliver a variable mixture of biologically active and inactive substances, not all of which are desirable or even known. Raw plant material is also more susceptible than extracts to possible contaminants such as fungi or bacteria. There is therefore a continuing need for improved delivery systems and improved products that do not involve smoking the crude Cannabis plant material in order to obtain the cannabinoid compounds for therapeutic purposes and clinical study.

Alternate delivery methods such as Cannabis oil vaporizers or cannabinoid patches such as those disclosed in U.S. Pat. No. 6,113,940 (2000) to Brooke et al. require clinical grade Cannabis extracts. The extraction and purification of Cannabis constituents has been performed by various methods, optionally combined with “isomerization” of the CBD in the extract to THC. Such extraction systems typically emphasize complete extraction of the valuable cannabinoids via methods such as Soxhlet extractors, prolonged reflux of the Cannabis in solvent or repeated extraction, optionally combined with washes and further purification. However, these processes have significant limitations in that total extraction does not allow for any initial enrichment of therapeutic compounds. Further processing, such as activated charcoal treatment, distillation under high vacuum, etc., can provide enriched extracts, but such increases labor, equipment and other processing costs.

In many applications there is a need for simple Cannabis extracts enriched in therapeutic cannabinoids as compared to total extracts. There is also a continuing need for improved processes for producing Cannabis extracts. There is also a need for simple Cannabis extracts of improved quality wherein the therapeutic effect is enhanced. There is also a need for such methods and products as an aid to improved delivery systems for Cannabis products.

### BRIEF SUMMARY OF THE INVENTION

The present invention provides for improved extracts of Cannabis plant material by utilizing a partial extraction wherein a solvent is allowed to contact the plant material for a period of time less than that needed to reach an equilibrium of dissolved materials in solvent. Such time-sensitive selective partial extraction advantageously provides for improved Cannabis extracts and oils. This technique may also be utilized with other plants having elevated levels of soluble therapeutic components in plant surface structures or near-surface structures.

In view of the disadvantages inherent in the known products and production methods, the present invention provides improved extracts and production processes. Still further objects and advantages of this invention will become more apparent from the following detailed description and appended claims. Before explaining the disclosed embodiments of the present invention in detail, it is to be understood that the invention is not limited in its application to the details of the particular products and methods illustrated, since the invention is capable of other embodiments which will be readily apparent to those skilled in the art. Also, the terminology used herein is for the purpose of description and not of limitation.

### DETAILED DESCRIPTION OF THE INVENTION

The present invention provides for the improved separation of cannabinoids from Cannabis plant material by controlling the duration of the saturation during the initial solvent extraction. Utilizing a comparatively short duration contact time provides for the preparation of extracts relatively deficient in the non-therapeutic compounds found in the plant material and relatively enriched in those therapeutic compounds, such as THC, found in surface microstructures such as lactifers, stalked and sessile glands including unicellular covering hairs, the trichomes and cystoliths, multicellular glandular hairs and resins released from those structures.

The present inventors have found that shorter solvent contact times are associated with higher-grade oil extract exhibiting less signs of high molecular weight tars and oils. This leads to Cannabis oil drug substance suitable for many clinical applications without further refinement.

Various compositions may be formulated depending on solvent choice and extraction time. While ethanol is generally preferred for therapeutic applications, other solvents that may be of use in various formulations and applications include acetone, acetonitrile, benzene, butanols, chloroform, cyclohexane, 1,2-dichloroethane, dichloromethane, diethyl ether, N,N-dimethylformamide, ethyl acetate, hexane, isopropanol, methanol, methylbutyl ketone, methylcyclohexane, pentane, propanols, tetrahydrofuran, toluene, and xylene and the other solvents and solvent systems known to the art. If ethanol is utilized as the extraction solvent, anhydrous absolute (200 proof) alcohol is preferable to 95% (190 proof). A non-denatured ethanol is preferable. If a denatured alcohol is utilized, a denaturant such as methanol or other substance with a boiling point lower than ethanol is preferred, and removal of said denaturant must be achieved through possible further refinement.

Changing the polarity and hydrophilicity of the extracting solvent systems can modify the ratio of components in the Cannabis oil. When using an alcohol as the primary extracting solvent, both polar and non-polar compounds are extracted. Initial or subsequent extraction with non-polar solvents such as hexane or diethyl ether leads to refined oils free from chlorophyll and sugars. However, the water-soluble components may contribute to the overall therapeutic effect and makeup of the volatile bouquet.

Unlike prior art processes, mechanical disruption (cutting, grinding or crushing) of the Cannabis plant material prior to extraction and/or agitation during extraction is not preferred. Such mechanical disruption and/or agitation tends to disrupt plant cells and increase solubilization of the less desirable non-therapeutic components. Seedless female plants are preferably utilized; where seeded material is utilized, the seeds are preferably removed while disrupting the plant material as little as is possible.

In general, the duration of the contact with solvent should be under three minutes (180 seconds). Preferably, the contact time should be under one minute (60 seconds). More preferably, the contact time should be 30 seconds or less for best results. For preparation of the highest quality extracts (“camalis oil”), contact time should be less than 5 seconds (preferably 0.01-4 seconds, more preferably 1-3 seconds, with about 2.5 seconds being optimum). With a 30 second extraction time utilizing absolute ethanol in accordance with Example 1 below, approximately 55-60% of the total soluble material is removed in the first pass. As solvent contact times are increased, the extracted, oil-like material becomes thicker; when utilizing ethanol as a solvent, increasing amounts of chlorophyll are extracted with increasing contact times. In general, extraction of more than ⅔ of the soluble material on the first pass is not desirable, with less than 60% being preferred.

Additional extraction(s) may be performed upon the plant material. This leads to Cannabis oil that may or may not need further refinement, depending upon application—such secondary extracts may be utilized as is for cooking, etc., or further refined for use in clinical applications.

Techniques known to the art such as steam distillation or activated charcoal filtration may optionally be utilized to further enrich the cannabinoid fraction of extracts; fractional distillation and column chromatography of the extracts can be used to isolate the major components of the Cannabis extracts, including the terpenes and sesqui-terpenes and major cannabinoid components including cannabidiol, cannabinol and delta-9-THC. The lower boiling terpenes and sesqui-terpenes are not psychoactive but may contribute to the essence and therapeutic value of ethanolic short duration contact extracts. Any CBD present in the extracts may optionally be isomerized to THC via methods such as reflux in an appropriate solvent with an acid such as sulfuric acid or p-toluenesulfonic acid.

As the active ingredients of the Cannabis plant are typically lipophilic, the oils containing the desired ingredients may be extracted and utilized in numerous delivery systems that are less harmful to the respiratory system. Or, if smoking or vaporization is the desired patient method of delivery, a material may be provided by the present invention in which a relatively large percent of the plant vegetable material and toxic byproducts of combustion are eliminated.

Formulations and delivery systems useful in the practice of the present invention include, by way of example but not of limitation, pills and encapsulations, herbal blends, tinctures, transdermal delivery patches, transcutaneous carriers, oils and gels, vaporizer formulations, eye drops, etc. Those methods known to those skilled in the art for increasing the bioavailability of lipophilic substances may also be utilized in conjunction with the extracts of the present invention. The drug substance extracts of the present invention may be favorably combined with other medicinal herbs or extracts such as Echinacea purpurea, Kava root powder, Ginkgo biloba, Salvia divinorum (Diviner's Sage), etc., optionally with excipients.

The present invention may also be applied to other medicinal herbs besides Cannabis that have surface or near-surface structures enriched in medicinal compounds (e.g., stinging nettles, etc.) and as a method of lessening the amount of non-therapeutic compounds in all medicinal herb extracts. As such, the present invention is intended to include all medicinal herbs to which it may be favorably applied.

### EXAMPLE 1

2.27 kg. (5 lb.) of dried medical grade Cannabis (seedless female flowering tops harvested in California, USA) was dried at 93° C. (200° F.) for 5 minutes to decarboxylate the cannabinoids (e.g., THC acid to THC). The material was then placed in a muslin cloth basket approximately 46 cm. (18 in.) in diameter and 61 cm. (24 in.) in height and extracted with 7.6 liters (2 gal.) anhydrous non-denatured ethanol, the ethanol being in contact with the Cannabis for about 30 seconds from the time the first of the ethanol was poured upon the Cannabis until most of the ethanol drained from the plant material. The Cannabis retained approximately 1.9 liters (0.5 gal.) and approximately 5.7 liters (1.5 gal.) of cannabinoid-enriched ethanol was recovered from the wet Cannabis. Vacuum evaporation of the solvent gave approximately 225 grams (˜10% of dry plant weight) of cannabinoid-enriched Cannabis oil.

### EXAMPLE 2

Approximately 60 ml (¼ cup) of powdered Kava Kava root powder was placed into a sterilized mortar and 10 ml (˜10.3 grams) of Cannabis oil dissolved in 10 ml of ethanol was slowly added and mixed until homogeneous while grinding with a pestle. The mixture was spread into a sterilized dish, covered with filter paper and placed on a heat pad at low heat for one hour. If still wet, the product was mixed and the heating repeated. Once the Kava and Cannabis oil mixture was found void of solvent, the material was transferred and ground fine with mortar and pestle. The resultant material was encapsulated into 75 “00” capsules.

It should be understood the foregoing detailed description is for purposes of illustration rather than limitation of the scope of protection accorded this invention, and therefore the description should be considered illustrative, not exhaustive. The scope of protection is to be measured as broadly as the invention permits. While the invention has been described in connection with preferred embodiments, it will be understood that there is no intention to limit the invention to those embodiments. On the contrary, it will be appreciated that those skilled in the art, upon attaining an understanding of the invention, may readily conceive of alterations to, modifications of, and equivalents to the preferred embodiments without departing from the principles of the invention, and it is intended to cover all these alternatives, modifications and equivalents. Accordingly, the scope of the present invention should be assessed as that of the appended claims and any equivalents falling within the true spirit and scope of the invention.

We claim:

1. A method for preparing Cannabis extracts comprising:
a) contacting Cannabis plant material with a solvent for an amount of time less than that necessary for the solvent to reach an equilibrium concentration of cannabinoids; and
b) separating the solvent and dissolved cannabinoids from the Cannabis plant material.

2. The method for preparing Cannabis extracts of claim 1 wherein the solvent is in contact with the Cannabis plant material for less than three minutes.

3. The method for preparing Cannabis extracts of claim 1 wherein the solvent is in contact with the Cannabis plant material for less than one minute.

4. The method for preparing Cannabis extracts of claim 1 wherein the solvent is in contact with the Cannabis plant material for less than 30 seconds.

5. The method for preparing Cannabis extracts of claim 1 wherein the solvent is in contact with the Cannabis plant material for less than 5 seconds.

6. The method for preparing Cannabis extracts of claim 1 wherein the solvent dissolves less than ⅔ of the cannabinoids.

7. The method for preparing Cannabis extracts of claim 1 wherein the solvent dissolves less than 60% of the cannabinoids.

8. The method for preparing Cannabis extracts of claim 1 wherein the solvent is selected from the group consisting of acetone, acetonitrile, benzene, butanols, chloroform, cyclohexane, 1,2-dichloroethane, dichloromethane, diethyl ether, N,N-dimethylformamide, ethanol, ethyl acetate, hexane, isopropanol, methanol, methylbutyl ketone, methylcyclohexane, pentane, propanols, tetrahydrofuran, toluene, xylene and combinations thereof.

9. The method for preparing Cannabis extracts of claim 1 wherein the solvent comprises undenatured ethanol.

10. The method for preparing Cannabis extracts of claim 9 wherein the undenatured ethanol is anhydrous.

11. The method for preparing Cannabis extracts of claim 1 comprising the additional step of isomerizing CBD to THC.

12. The method for preparing Cannabis extracts of claim 1 comprising the additional step of removing the solvent.

13. A Cannabis extract comprising an extract prepared by contacting Cannabis plant material with a solvent for a length of time less than that necessary for all soluble components to dissolve and separating the Cannabis plant material from the solvent and cannabinoids dissolved therein.

14. The Cannabis extract of claim 13 wherein the solvent is in contact with the Cannabis plant material for less than three minutes.

15. The Cannabis extract of claim 13 wherein the solvent is in contact with the Cannabis plant material for less than one minute.

16. The Cannabis extract of claim 13 wherein the solvent is in contact with the Cannabis plant material for less than 30 seconds.

17. The Cannabis extract of claim 13 wherein the solvent is in contact with the Cannabis plant material for less than 5 seconds.

18. The Cannabis extract of claim 13 wherein the solvent dissolves less than ⅔ of the cannabinoids.

19. The Cannabis extract of claim 13 wherein the solvent dissolves less than 60% of the cannabinoids.

20. The Cannabis extract of claim 13 wherein the solvent is selected from the group consisting of acetone, acetonitrile, benzene, butanols, chloroform, cyclohexane, 1,2-dichloroethane, dichloromethane, diethyl ether, N,N-dimethylformamide, ethanol, ethyl acetate, hexane, isopropanol, methanol, methylbutyl ketone, methylcyclohexane, pentane, propanols, tetrahydrofuran, toluene, xylene and combinations thereof.

21. The Cannabis extract of claim 13 wherein the solvent comprises undenatured ethanol.

22. The Cannabis extract of claim 21 wherein the undenatured ethanol is anhydrous.

23. The method for preparing Cannabis extracts of claim 13 wherein CBD is isomerized to THC.

24. The Cannabis extract of claim 13 wherein the solvent is removed from the cannabinoids.

25. A method for preparing extracts of medicinal plants comprising contacting medicinal plant material with a solvent for an amount of time less than that necessary for the solvent to reach an equilibrium concentration of dissolved medicinal components.
